(19) United States
(12) Patent Application Publication
(10) Pub. No.: US 2010/0261984 A1
(43) Pub. Date: Oct. 14, 2010

(54) Physiology sensing apparatus with navigation capability

(75) Inventors: Ming-Jye Tsai, Xinpu Town (TW); Yung-Yu Chen, Xinyuan Shiang (TW); Shi-Hao Wang, Taichung City (TW)

Correspondence Address: WPAT, PC;INTELLECTUAL PROPERTY ATTORNEYS, 7225 BEVERLY ST., ANNANDALE, VA, 22003, US

(73) Assignee: Industrial Technology Research Institute

(21) Appl. No.: 11/487,497

(22) Filed: Jul. 17, 2006

(30) Foreign Application Priority Data
Dec. 22, 2005 (TW) 094145783

(51) Int. Cl. A61B 5/01; A61B 5/00; A61B 5/053; A61B 5/024; G01S 19/42

(52) U.S. Cl. 600/306; 600/300; 600/481; 600/549

(57) ABSTRACT

A physiology sensing apparatus with navigation capability is disclosed, which integrates therein a GPS module, a magnetic sensor, an inertial sensing module, and a bio sensing module so as to acquire signals relating to a user's coordinate position, physical condition, body movement, and so on, and then the acquired signals can be purified by filter devices so as to reduce the interference of noise, thereafter, processes the filtered signal by a signal processor and an alarming device so as to simultaneously obtain the information of user's location, physical condition, and whether the user is in danger or having an accident.

### FIELD OF THE INVENTION

The present invention related to a physical sensing apparatus with navigation capability, and more particularly, to a physical sensing apparatus capable of utilizing a GPS module, a magnetic sensing module, an inertial sensing module and a bio sensing module for obtaining the information of a user's position, physical status, and whether the user is in danger or having an accident.

### BACKGROUND OF THE INVENTION

Global Positioning System (GPS) is generally referred to as a worldwide radio-navigation system formed from a constellation of 24 satellites and their ground stations, whereas the most obvious application of a GPS device is the simple determination of a user's position or location. It is therefore the operation of a GPS device is restricted by its ability of receiving signals from those satellites and ground stations. As a result these GPS devices are subject to occlusions and numerous interferences and noise sources. For instance, once a user of a GPS device is inside a building or enters an urban area, the accuracy of the GPS device will be severely affected by shielding effect of its surrounding that, in a worst circumstance, the GPS device is incapable of receiving any signals from those satellites and ground stations. Therefore, additional ground stations or auxiliary orientation devices are required to assist the GPS device to overcome such shielding effect. Nowadays, as the advance of inertial sensing technology, inertial sensors are desirable for general motion sensing because they operate regardless of external references, and can be used for compensating the GPS devices as they are subjecting to interferences of terrain and noises. Therefore, there are already many studies relating to the integration of GPS device, inertial sensing devices and magnetic sensing device for tracking and positioning.

In the prior-art technologies as disclosed in U.S. Pat. No. 6,805,844, U.S. Pat. No. 6,845,323, U.S. Pat. No. 6,529,827, and U.S. Pat. No. 6,834,150, many resolutions regarding to the positioning of a person had be provided. However, there is no disclosure of any integrated method for accurately and rapidly determining a user's coordinate position, physical condition, and motion statuses simultaneously.

Therefore, it is in need of a physiology sensing apparatus with navigation capability, which integrates means of positioning, bio sensing and accident evaluating for providing and recording signals relating to a user's coordinate position, physical condition, and body movement in real time while detecting whether the user is in danger or having an accident.

### SUMMARY OF THE INVENTION

It is the primary object of the present invention to provide a monitoring apparatus integrating means of global positioning, bio sensing and inertial sensing, which can be adapted to adhere itself to the bodies of an aged person, child, or patient for detecting his/her location, physical condition, and moving statuses.

It is another object of the invention to provide an apparatus capable of detecting a user's location, physical condition, and body movement, while utilizing a communication module to transmit the detected information to a displaying device or a remote control center for facilitating the condition of the user to be monitored constantly.

To achieve the above objects, the present invention provide a physiology sensing apparatus with navigation capability, which comprises a GPS module, a magnetic sensing module, an inertial sensing module, a bio sensing module, a filter device, and a signal processor, and an alarming device.

Wherein, the GPS module is used for receiving satellite positioning signals to issue a first orientation signal accordingly; the magnetic sensing module is used for measuring the variation of geomagnetic strength to issue a second orientation signal accordingly; the inertial sensing module is used for detecting a specific body movement of a user to issue a gesture signal accordingly and it is also responding to a reallocation of the user to issue a third orientation signal accordingly; the bio sensing module is used for sensing the physical condition of the user to issue a bio signal accordingly; the filter device is used for filtering the noises of the first orientation signal, the second orientation signal, the third orientation signal, the gesture signal and the bio signal; the signal processor is used for processing an operation upon the filtered signals to output a digital signal accordingly; and the alarming device is used to evaluate whether the received digital signal is abnormal and to issue an alarming signal if so.

Preferably, the physiology sensing apparatus with navigation capability further comprises a communication module, which is used for transmitting the digital signal and the alarming signal to a remote control center.

Preferably, the physiology sensing apparatus with navigation capability further comprises a displaying device, which is used for receiving the digital signal so as to display information of the location, physical condition, and body movement of the user while being enabled to receive the alarming signal for issuing an audio/video alarm, such as flashing light or beeping.

In a preferred embodiment of the invention, the magnetic sensing module can be an electrical compass; and the inertial sensing module further comprises at least an inertial sensor, which can be a device selected from the group consisting of an accelerometer, a gyroscope and a level; and the bio sensing module further comprises at least a bio sensor, which can be a device selected from the group consisting of a heart-beat sensor, a blood oxygen saturation meter, a blood flow index sensor, a skin resistance meter, a temperature sensor, and a moisture sensor.

Moreover, the digital signal contains information relating to the user's location, motion and body movement and physical condition, that the digital signal is determined as abnormal in the evaluation of the alarming device as one of the following conditions is established: (1) the received digital signal is larger than a predetermined maximum threshold; (2) the received digital signal is smaller than a predetermined minimum threshold; and (3) the difference between the two successive received digital signals is larger than a specific predetermined value.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 is a block diagram of a physiology sensing apparatus with navigation capability according to a first preferred embodiment of the invention.

FIG. 2 is a block diagram of a physiology sensing apparatus with navigation capability according to a second preferred embodiment of the invention.

### DESCRIPTION OF THE PREFERRED EMBODIMENT

For your esteemed members of reviewing committee to further understand and recognize the fulfilled functions and structural characteristics of the invention, several preferable embodiments cooperating with detailed description are presented as the follows.

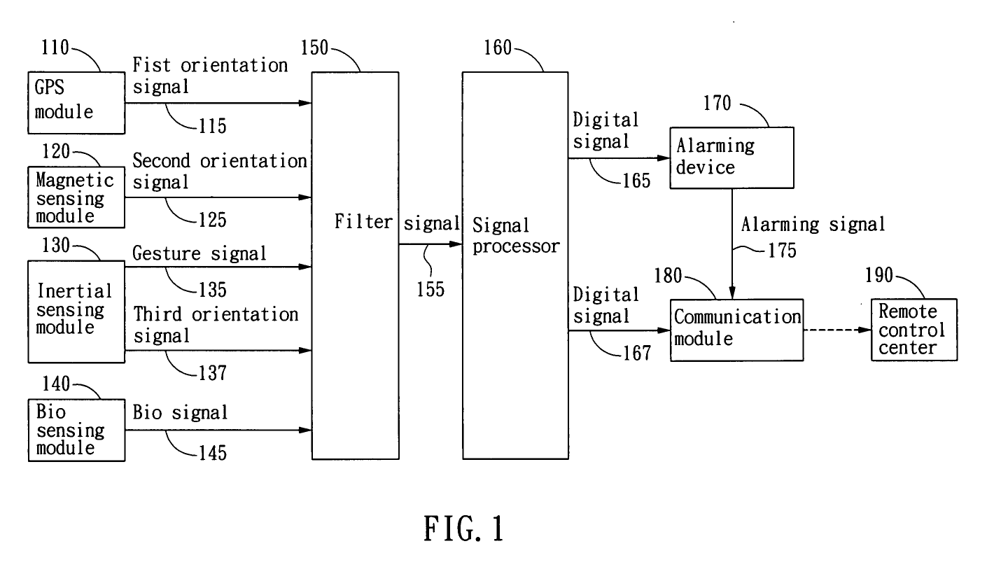

Please refer to FIG. 1, which is a block diagram of a physiology sensing apparatus with navigation capability according to a first preferred embodiment of the invention. The physiology sensing apparatus with navigation capability of FIG. 1 is comprised of a GPS module 110, a magnetic sensing module 120, an inertial sensing module 130, a bio sensing module 140, a filter device 150, and a signal processor 160, an alarming device 170 and a communication module 180.

Wherein, the GPS module 110 is used for receiving satellite positioning signals to issue a first orientation signal 115 accordingly; the magnetic sensing module 120 is used for measuring the variation of geomagnetic strength to issue a second orientation signal 125 accordingly; the inertial sensing module 130 is used for detecting a specific gesture of a user to issue a gesture signal 135 accordingly and it is also responding to a reallocation of the user to issue a third orientation signal 137 accordingly; the bio sensing module 140 is used for sensing the physical condition of the user to issue a bio signal 145 accordingly; the filter device 150 is used to perform a noise filtering operation upon the first orientation signal 115, the second orientation signal 125, the third orientation signal 137, the gesture signal 135 and the bio signal 145; the signal processor 160 is used for processing an operation upon the filtered signal 155 to output two digital signals 165, 167 accordingly; the alarming device 170 is used to evaluate whether the received digital signal 165 is abnormal and to issue an alarming signal 175 if so; and the communication module 180 is used for transmitting the digital signal 167 and the alarming signal 175 to a remote control center 190.

By the first orientation signal 115, the second orientation signal 125 and the third orientation signal 137 respectively provided by the GPS module 110, the magnetic sensing module 120 and the inertial sensing module, the coordinates of the user's location can be accurately detected. Since the magnetic sensing module 120 can be an electrical compass or other electronic devices capable of providing locality information utilizing magnetic sensing technology, the locality of a user can be detected and measured by the cooperation of the GPS module 110 and the magnetic sensing module 120 even when the user is motionless or moving slowly. Moreover, when the GPS module 110 fails to receive satellite positioning signals, i.e. when there is no first orientation signal 115 available, the third orientation signal 137 of the inertial sensing module 130 can be used to provide information relating to the reallocation of the user so that the locality of the user still can be detected.

The inertial sensing module 130 can be a device comprised of accelerometers. gyroscopes and levels, which not only is capable of providing relocating information of the user, but also is capable of detecting and recording data relating to a specific movement of the user, such as power, acceleration, angular acceleration, velocity, reallocation, and so on. For instance, when the user is tripped to fall down, a forward inclining force is generated momentarily and the inertial sensing module 130 is enabled to record the data relating to this movement while outputting a signal accordingly.

The bio sensing module 140 can be a device composed of heart-beat sensor, a blood oxygen saturation meter, a blood flow index sensor, a skin resistance meter, a temperature sensor, and a moisture sensor and the like, which is capable of detecting and recording data relating to physical condition of the user while outputting a signal according accordingly.

Since the first and the second orientation signals 115, 125 are vulnerable to the interferences of ambient environment, the filter device 150 is used to filter out the noises of the first and the second orientation signals 115, 125. In addition, the filter device 150 also can be used to filter out the noise of the gesture signal 135, the third orientation signal 137 and the bio signal 145 so that much more accurate data relating to the user can be acquired therefrom. The filter device can be implemented as required, which can be comprised of a plurality of filters for eliminating noises of signals inputted thereto in respective.

The signal processor 160 is used for processing an operation upon the filtered signals 155 to output two digital signals 165, 167 respectively to the alarming device 170 and the communication module 180.

The alarming device 170 is used to perform an evaluation upon the digital signal 165 for determining whether the digital signal 165 is indicating any abnormality and issuing an alarming signal 175 if so. That is, the digital signal 165 is indicating any abnormality as one of the following conditions is established: (1) the digital signal 165 is larger than a predetermined maximum threshold; (2) the digital signal is smaller than a predetermined minimum threshold; and (3) the difference between the two successive received digital signals is larger than a predetermined value. The abnormality of the digital signal 165 represents the user in not in good shape or having an accident. For instance, when the voltage representing body temperature is detected and is higher than a predetermined maximum threshold, which represents the body temperature of the user is higher than 38° C., the alarming signal 175 is issued; or when voltages representing acceleration are detected and the voltage difference between two successive received sampling points is larger than a predetermined threshold, which may indicate that the users might be running, squatting or tripping. However, by combining the aforesaid overly high voltage of acceleration with other detected information relating to direction of the acceleration, location, and the physical condition of the user, an evaluation can be made to determine whether the user is having an accident. It is noted that the above descriptions are only embodiments of the invention and is not limited thereby.

The communication module 180 is used for transmitting the digital signal 167 and the alarming signal 175 to a remote control center 190 for facilitating the condition of the user to be monitored constantly, which is especially important for aged persons, children, or patients. Generally, the communication module 180 achieves the transmission by a wireless means. However, other communication specifications and protocols can be adopted thereby as required.

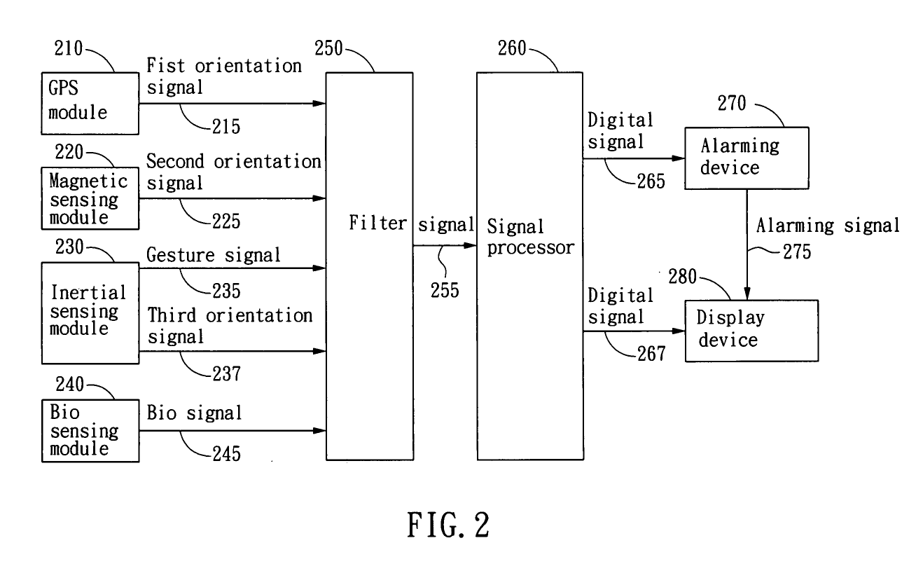

Please refer to FIG. 2, which is a block diagram of a physiology sensing apparatus with navigation capability according to a second preferred embodiment of the invention. The physiology sensing apparatus with navigation capability of FIG. 2 is comprised of a GPS module 210, a magnetic sensing module 220, an inertial sensing module 230, a bio sensing module 240, a filter device 250, and a signal processor 260, an alarming device 270 and a displaying device 280.

Wherein, the GPS module 210 is used for receiving satellite positioning signals to issue a first orientation signal 215 accordingly; the magnetic sensing module 220 is used for measuring the variation of geomagnetic strength to issue a second orientation signal 225 accordingly; the inertial sensing module 230 is used for detecting a specific gesture of a user of the physiology sensing apparatus to issue a gesture signal 235 accordingly while responding to a reallocation of the user to issue a third orientation signal 237 accordingly; the bio sensing module 240 is used for sensing the physical condition of the user to issue a bio signal 245 accordingly; the filter device 250 is used to perform a noise filtering operation upon the first orientation signal 215, the second orientation signal 225, the third orientation signal 237, the gesture signal 235 and the bio signal 245; the signal processor 260 is used for processing an operation upon the filtered signal 255 to output two digital signals 265, 267 accordingly; the alarming device 270 is used to perform an evaluation upon the digital signal 265 for determining whether the value of the digital signal 265 is abnormal and issuing an alarming signal 275 if so; and the display device 280 is used for receiving the digital signal 267 and the alarming signal 275 so as to display information of the location, physical condition, motion statuses of the user while enabling the same to issue an alarm.

The difference between the first and the second embodiments of the invention is that, in the second embodiment, the information of the location, physical condition, and body movement of the user can be displayed directly on the displaying device 280. The displaying device can be a small-sized liquid crystal display panel or other electric circuits with displaying ability, which is able to display a flashing light or issue a beeping sound for alerting the user while enabling the user to aware of his/her locality and physical conditions in a constant manner.

To sum up, the present invention provides a physiology sensing apparatus with navigation capability, which integrates therein a GPS module, a magnetic sensor, an inertial sensing module and a bio sensing module so as to acquire signals relating to a user's coordinate position, physical condition, and body movement, and so on, and then the acquired signals can be purified by filter devices so as to reduce the interference of noise, thereafter, processes the filtered signal by a signal processor and an alarming device so as to simultaneously obtain the information of user's position, physical status, and whether the user is in danger or having an accident.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

1. A physiology sensing apparatus with navigation capability, comprising:
a signal processor processing a filtered signal and outputting a digital signal;
a filter device physically coupled to the signal processor, and issuing the filtered signal to the signal processor;
a GPS module coupled to the filter device, receiving satellite positioning signals and issuing a first orientation signal to the filter device;
a magnetic sensing module, coupled to the filter device, measuring the variations of geomagnetic strength and issuing a second orientation signal to the filter device;
an inertial sensing module, coupled to the filter device, issuing a gesture signal to the filter device wherein the inertial sensing module generates the gesture signal by detecting a specific body gesture of a user, and generating a third orientation signal in response to a reallocation of the user;
a bio sensing module coupled to the filter device, sensing a physical status of the user and issuing a bio signal to the filter device; and
an alarming device coupled to the signal processor, and receiving the digital signal from the signal processor;
wherein the filter device issues the filtered signal by filtering noises of a first orientation signal, a second orientation signal, a third orientation signal, a gesture signal, and a bio signal; and the alarming device issues an alarm signal after evaluating the digital signal.

2. The physiology sensing apparatus of claim 1, further comprising:
a communication module coupled to the signal processor, transmitting the digital signal outputted by the signal processor to a remote control center.

3. The physiology sensing apparatus of claim 1, further comprising:
a communication module coupled to the alarming device, transmitting the alarming signal outputted by the alarming device to a remote control center.

4. The physiology sensing apparatus of claim 1, further comprising:
a displaying device coupled to the signal processor, for receiving the digital signal from the signal processor to display information of location, physical condition, and body gesture of the user.

5. The physiology sensing apparatus of claim 1, further comprising:
a displaying device coupled to the alarming device, receiving the alarming signal to issue an alarm.

6. The physiology sensing apparatus of claim 5, wherein the alarm is an audio/video alert providing a flashing light or a beeping sound.

7. The physiology sensing apparatus of claim 1, wherein magnetic sensing module is an electrical compass.

8. The physiology sensing apparatus of claim 1, wherein the inertial sensing module is enabled to issue the third orientation signal while the GPS module fails to receive satellite positioning signals.

9. The physiology sensing apparatus of claim 1, wherein the inertial sensing module further comprises at least an inertial sensor.

10. The physiology sensing apparatus of claim 9, wherein the inertial sensor is an accelerometer.

11. The physiology sensing apparatus of claim 9, wherein the inertial sensor is a gyroscope.

12. The physiology sensing apparatus of claim 9, wherein the inertial sensor is a level.

13. The physiology sensing apparatus of claim 1, wherein the bio sensing module further comprises at least a bio sensor.

14. The physiology sensing apparatus of claim 13, wherein the bio sensor is a device selected from the group consisting of a heart-beat sensor, a blood oxygen saturation meter, a blood flow index sensor, a skin resistance meter, a temperature sensor, and a moisture sensor.

15. The physiology sensing apparatus of claim 1, wherein the digital signal contains information relating to the user's location.

16. The physiology sensing apparatus of claim 1, wherein the digital signal contains information relating to the user's body movement.

17. The physiology sensing apparatus of claim 1, wherein the digital signal contains information relating to the user's physical condition.

18. The physiology sensing apparatus of claim 1, wherein the alarming signal is issued as the digital signal is larger than a predetermined maximum threshold.

19. The physiology sensing apparatus of claim 1, wherein the alarming signal is issued as the digital signal is smaller than a predetermined minimum threshold.

20. The physiology sensing apparatus of claim 1, wherein the alarming signal is issued as difference between two successive received digital signals is larger than a predetermined value.
